(19) United States
(12) Patent Application Publication
(10) Pub. No.: US 2024/0032513 A1
(43) Pub. Date: Feb. 1, 2024

(54) Materials for Tumor Inoculation in Murine Mouse Models and Uses Thereof

(71) Applicant: The Board of Trustees of the Leland Stanford Junior University, Stanford, CA (US)

(72) Inventors: Abigail Kate Grosskopf, Chadds Ford, PA (US); Eric Andrew Appel, Stanford, CA (US); Santiago Correa, Palo Alto, CA (US)

(21) Appl. No.: 18/022,234

(22) PCT Filed: Oct. 20, 2021
(86) PCT No.: PCT/US2021/055760

§ 371 Date: Feb. 20, 2023

Related U.S. Application Data
(60) Provisional application No. 63/094,716, filed on Oct. 21, 2020.

(51) Int. Cl. A01K 67/027; A61L 27/52; A61L 27/38

(52) U.S. Cl. CPC A01K 67/0271; A61L 27/52; A61L 27/3804; A01K 2267/0331; A01K 2207/12; A01K 2227/105; A61L 2400/06

(57) ABSTRACT

Preclinical cancer research is heavily dependent on allograft and xenograft models, but current approaches to tumor inoculation yield inconsistent tumor formation and growth, ultimately wasting valuable resources (e.g., animals, time, and money) and limiting experimental progress. A method and kit for tumor inoculation is disclosed using self-assembled hydrogels to reliably generate tumors with low variance in growth. The observed reduction in model variance enables smaller animal cohorts, improved effect observation and higher-powered studies.

### FIELD OF THE INVENTION

This invention relates to methods and kits for tumor inoculation in cancer studies and modelling.

### BACKGROUND OF THE INVENTION

A reproducibility crisis in preclinical research has contributed to disappointing outcomes in clinical trials. In response, significant effort has gone into developing animal models that better recapitulate human disease with the hope of improving the predictive power of preclinical research. Yet, little attention has been paid to improving consistency of animal models or developing facile, lab transferable techniques. As a result, inconsistent animal models are endemic and complicate comparisons of data from different labs, within labs, and even within individual experiments. Subcutaneous allograft and xenograft flank models are among the most common models used for preclinical cancer research, particularly for immunooncology. These models involve injecting cancerous cells dispersed in liquid buffered saline subcutaneously on the flank of animals and waiting for tumors to form (FIG. 1A).

Variance in tumor formation necessitates researchers use more animals to conduct sufficiently powered studies, and the chance of failed tumor formation further inflates animal cohort sizes (FIG. 1A). Indeed, technical challenges typically lead to upwards of 30% of inoculated mice failing to form tumors. Preclinical cancer studies can involve hundreds of mice, so significant resources, time, and animals can be wasted due to the severe inefficiencies present in current model protocols. Since mice with abnormal or late-growing tumors must be euthanized without having contributed useful data to a study, significant ethical concerns are raised by the unnecessary overuse of research animals. Reliable and reproducible models would dramatically reduce the number of mice needed and increase the rate at which discoveries can be made by improving study power.

Current approaches to cancer cell inoculation suffer from two primary drawbacks:
(i) inoculation is poorly reproducible animal-to-animal because of cell agglomeration or settling in the syringe prior to injection, and
(ii) implanted cells lack extracellular matrix providing critical biochemical and biophysical cues.

Previous reports indicate that cancer cells encapsulated in basement membrane extract (BME) form tumors more rapidly in murine models when compared to cells suspended in saline. While these results are promising, BME is a poorly defined solution derived from Engelbreth-Holm-Swarm mouse sarcoma, thereby suffering from considerable batch-to-batch variability, and its temperature-dependent gelation introduces technical difficulties in avoiding premature gelation within the syringe. While BME offers tumorigenic factors like Laminin and Type IV Collagen, the uncertainty of its contents and technically challenging handling provide good reason for researchers to avoid it. There has been increasing interest in the development of alternative, molecularly defined hydrogel scaffolds for applications in tissue engineering. Injectable hydrogels have been developed that can enhance therapeutic cell administration by protecting cells from mechanical forces during injection, enabling homogeneous injections and enhancing cell retention at the injection site1. The present invention advances the art with technology of a self-assembled hydrogel for controlled encapsulation and delivery of cancer cells that improves the reproducibility of tumor formation.

### SUMMARY OF THE INVENTION

### Definitions

BME (Basement Membrane Extract) is defined as a soluble extract of basement membrane components derived from the Engelbreth-Holm-Swarm (EHS) tumor containing collagens, regularly arrayed sulfated macromolecules, laminin, a high content of glycosylated molecules, and many other components.
B16F10 melanoma model is a commonly utilized murine model based on metastatic murine melanoma. It can be administered subcutaneously to form tumors or administered peritumorally or intravenously.

In this invention, a self-assembled hydrogel material, methods and a kit have been developed for controlled encapsulation and delivery of cancer cells that addresses the reproducibility of tumor formation. With this approach, tumors form with lower variance in size, drastically improving statistics for large preclinical cancer studies.

In one embodiment, the invention is a tumor inoculation kit for developing a cancer model. The kit has solutions to form a self-assembled hydrogel, the cancer cells, a mixer for mixing the solutions and the cancer cells and forming self-assembled hydrogels encapsulating the cancer cells, and a syringe for injecting the cancer cells encapsulated by the self-assembled hydrogels.

In another embodiment, the invention is a method of tumor inoculation for developing a cancer model. The method provides solutions to form a self-assembled hydrogel, provides, uses or has the cancer cells, mixes the solutions and the cancer cells to form self-assembled hydrogels encapsulating the cancer cell, and injects the cancer cells encapsulated by the self-assembled hydrogels into tissue.

The key solutions to form the self-assembled hydrogel are Alginate and Calcium Sulfate. In one example, the Alginate ranges from 0.5 to 2.5 wt % and the Calcium Sulfate ranges from 7 mM (or is another example 0 mM) to 15 mM. More specifically, Alginate is about 1 wt % and the Calcium Sulfate is about 10 mM. The cancer cells could be human or animal cancer cells. The cancer cells could also be a mixture of different types of cancer cells.

Cells other than cancer cells could be added to the kit or provided in the method. Examples of such cells are Vascular Endothelial Cells, Pericytes, Adipocytes, Fibroblasts, Bone-Marrow Mesenchymal Stromal Cells, or a combination thereof.

Extracellular Matrix Components (ECMs) could be added to the hydrogel to form the self-assembled hydrogel. Examples of ECMs are Laminin, Hyaluronic Acid, High Molecular Weight Hyaluronic Acid, Collagen I, Collagen II, Collagen III, Collagen IV, Collagen XVIII, Heparin, Heparin Sulfate, Fibronectin, Vitronectin, Gelatin, Elastin, Tenascin, Tenascin-C, Matrix Metalloproteinases, Basement Membrane Mixtures, or a combination thereof.

Growth factors could be added to the hydrogel to form the self-assembled hydrogel. Examples of growth factors are Fibroblast Growth Factors (FGF), Vascular Endothelial Growth Factor (VEGF), Platelet-derived growth factor (PDGF), stromal-derived factor-1 (SDF-1), TGF, tumor growth factor; TNF, tumor necrosis factor, or a combination thereof.

The self-assembled hydrogel useful for injection has a yield stress above 10 Pa. In another embodiment, the self-assembled hydrogel useful for injection has a yield stress ranging from 20 Pa to 60 Pa or 10 Pa to 60 Pa with a measured elastic storage modulus (G′) of 50-1000 Pa at 10 rad/s.

Furthermore, the self-assembled hydrogels useful for injection define a hydrogel volume ranging from 2 to 200 microliters in which the cancer cells are defined by a number of cancer cells within the hydrogel volume where the number of cancer cells ranging from 200,000 to 1,000,000.

In still another example, rheological modifiers could be added to modify rheological properties of the self-assembled hydrogel. For example, to modify the hydrogel's stress relaxation. Stress relaxation may enhance tumor growth.

Examples provided for the mixer is a dual syringe mixer distinguishing two connectable syringes for fluidably mixing the solutions and the cancer cells. Likewise, for this dual syringe mixer can be used for mixing the solutions and the cancer cells.

Embodiments of the invention can be used, not only as a method, but also as a kit that contains the hydrogel formulation for preclinical researchers to use in labs and companies that do cancer research.

Embodiments of the invention may also be used to alter and modulate the tumor microenvironment. For example, it could be used to help control different cell type interactions through co-encapulsulation in the hydrogel niche. It could be used to help cells gain enhanced exposure to a co-encapsulated growth factors that are slowly diffusing in the hydrogel. The gel's modular nature allows for control over the model tumor microenvironment.

### BRIEF DESCRIPTION OF THE DRAWINGS

FIGS. 1A-F show according to an exemplary embodiment of the invention self-assembled hydrogels for preclinical tumor models. FIG. 1A shows a current method of forming syngeneic subcutaneous tumors by injecting cancerous cells in liquid saline. FIG. 1B shows encapsulation process of B16F10 cells in self-assembled hydrogels and the benefits of cell delivery using hydrogels. FIG. 1C shows components that form self-assembled hydrogels including cancer cells, alginate, calcium, and extracellular matrix-mimicking biopolymers. FIG. 1D shows a dual syringe mixing strategy to formulate self-assembled hydrogels and encapsulate cells. A polymer solution containing alginate and ECM additives is loaded in one syringe (right) and cells and calcium sulfate are loaded into the second syringe (left). Blue dye (here in grey scale) is included to aid visualization for this demonstration. The two solutions are mixed with a female-female dual-syringe mixer by pumping the material back and forth for 30 seconds to form a robust hydrogel pre-loaded into a syringe ready for administration. FIG. 1E shows injection of self-assembled hydrogels into a hydrogel depot through syringe needles (25 G). FIG. IF shows oscillatory Shear Temperature ramp (w=10 rad/s, y=1%).

FIGS. 2A-J shows according to an exemplary embodiment of the invention syngeneic tumor model evaluation. FIGS. 2A-E show tumor area measurements over time following inoculation with various formulations containing encapsulated B16F10 cells (n=9 or 10 tumors). Average tumor area and standard deviation, and the corresponding resulting % CV. Saline exceeded a 100% CV on days 10 and 13. Gray lines included to guide the eye for better comparison. FIGS. 2F-J show histology images of tumors excised on day 15 following inoculation with hematoxylin and eosin staining. All scale bars represent 2 mm.

FIGS. 3A-F show according to an exemplary embodiment of the invention statistical evaluation of tumor growth, treatment, and power. FIG. 3A shows percentage of mice that formed palpable tumors during the 15-day experiment. FIG. 3B shows treatment day analysis from interpolation assuming exponential growth. Proportion treated from each group over time. FIG. 3C shows individual points (representing each mouse's treatment day) plotted with average and standard deviation. FIG. 3D shows power analysis predicting the number of mice per group needed based off the experimental data obtained with 80% power. The Statistical toolbox in Matlab was used for power calculations, specifically the sampsizepwr function with two-sided t-tests. The average and distribution of the day that each formulation surpassed 100 mm² were used in the power analysis. FIG. 3E shows power analyses predicting the effect of the tumor area % CV on the number of mice needed per group to obtain 80% power. Dotted lines represent % CV found from experimental formulations. Shading represents the area encompassing the 95% confidence intervals on the % CV values. The high estimate of the 95% confidence interval for both BME and saline exceed 50%. FIG. 3F shows power analyses predicting the effect of tumor % CV on the power of the study with 10 mice per group. The dotted colored lines represent the %CV found from experimental formulations. Shading represents the area encompassing the 95% confidence intervals on the % CV values. The high estimate of the 95% confidence interval for both BME and saline exceeds 50%.

FIGS. 4A-C show according to an exemplary embodiment of the invention in FIG. 4A amplitude sweep (w=10 rad/s) for all formulations at 37 degrees Celsius. All hydrogel formulations show a similar stiffness, a similar solid-like response, and a similar range of linear viscoelasticity across the measured strains. BME is less stiff, but yields at a similar strain. FIG. 4B, frequency sweep (strain=1%) for all formulations at 37 degrees Celsius. All hydrogel formulations show a similar stiffness, a similar solid-like response, and a similar range of linear viscoelasticity across the measured frequencies. BME is less stiff, but demonstrates a similar solid-like response. FIG. 4C flow sweep for Alginate and Alg-HA formulations at 25 degrees Celsius. Both formulations show a yield stress of approximately 20 Pa.

FIGS. 5A-B show according to an exemplary embodiment of the invention in FIG. 5A relative viability of B16F10 cells in various formulations after 1 day in culture after seeding 5000 cells. Luminescent signal is linear to the cell number. FIG. 5B, relative viability of EG7 cells after seeding 10,000 cells after 1 day in culture in various formulations. Error is reported as non-significant if p<0.05.

FIGS. 6A-C show according to an exemplary embodiment of the invention in vivo imaging over time of mice with luminescent tumors forming. Tumors were inoculated with cells dispersed in saline, BME, Alg, Alg-Lam and Alg-HA. FIG. 6A, saline (left, light purple) and BME (right, dark purple) groups. FIG. 6B, Alginate-Laminin (left, medium green) and Alginate (right, light green) groups. FIG. 6C, Alginate-Hyaluronic Acid (center, dark green) group. Colors in these figures have been converted to grey scale.

FIG. 7 shows according to an exemplary embodiment of the invention the average total flux in region of interest surrounding the tumor of interest and standard deviation from in vivo imaging on various days directly after inoculation for all experimental groups.

FIGS. 8A-B show according to an exemplary embodiment of the invention in FIG. 8A in vivo degradation of fluorescent alginate measured using an in vivo imaging system for all hydrogel groups. B16F10 cells were encapsulated and co-injected in all groups as in previous studies. Colors in these figures have been converted to grey scale. FIG. 8B, average total flux and standard deviation from the region of interest surrounding the injection over time. A one-phase exponential decay was fitted to each curve and a half-life was computed for each group as shown on the graph.

FIG. 9 shows according to an exemplary embodiment of the invention % CV of tumor area for all groups: saline, BME, Alg, Alg-Lam and Alg-HA. Same data as FIG. 5A plotted on one graph to show direct comparisons. Saline exceeded a 100% CV on Days 10 and 13.

FIG. 10 shows according to an exemplary embodiment of the invention P-values calculated with two-sided t-tests from analysis of tumor areas for all groups (FIGS. 5A-B).

FIG. 11 shows according to an exemplary embodiment of the invention additional histology images with Hemotoxylin and Eosin staining from Day 15 for tumors formed in saline. Columns represent a single tumor, and rows represent a single magnification. Saline tumors were smaller than other groups. 2× magnification the scale bars represent 2 mm. 4× magnification the scale bars represent 1 mm. 10× magnification the scale bars represent 400 micrometers.

FIG. 12 shows according to an exemplary embodiment of the invention additional histology images with Hemotoxylin and Eosin staining from Day 15 for tumors formed in BME. Columns represent a single tumor, and rows represent a single magnification. BME formed the largest tumors of all groups. 2× magnification the scale bars represent 2 mm. 4× magnification the scale bars represent 1 mm. 10× magnification the scale bars represent 400 micrometers.

FIG. 13 shows according to an exemplary embodiment of the invention additional histology images with Hemotoxylin and Eosin staining from Day 15 for tumors formed in Alginate. Microscopic examination by a pathologist confirmed that the melanomas demonstrated identical histomorphologic features across all formulations. There is amorphous, pale eosinophilic material additionally present in some tumors formed in gels that may represent residual gel material. This material is designated by black arrows. Upon evaluation by a blinded pathologist, these tumors do not appear morphologically different than tumors formed in BME and saline. These tumors formed in alginate were smaller than the other gel groups. Columns represent a single tumor, and rows represent a single magnification. 2× magnification the scale bars represent 2 mm. 4× magnification the scale bars represent 1 mm. 10× magnification the scale bars represent 400 micrometers.

FIG. 14 shows according to an exemplary embodiment of the invention additional histology images with Hemotoxylin and Eosin staining from Day 15 for tumors formed in Alginate-Laminin. There is amorphous, pale eosinophilic material additionally present in some tumors formed in gels that may represent residual gel material. This material is designated by black arrows. Upon evaluation by a blinded pathologist, these tumors do not appear morphologically different than tumors formed in BME and saline. Columns represent a single tumor, and rows represent a single magnification. 2× magnification the scale bars represent 2 mm. 4× magnification the scale bars represent 1 mm. 10× magnification the scale bars represent 400 micrometers.

FIG. 15 shows according to an exemplary embodiment of the invention additional histology images with Hemotoxylin and Eosin staining from Day 15 for tumors formed in Alginate-Hyaluronic Acid. Upon evaluation by a blinded pathologist, these tumors do not appear morphologically different than tumors formed in BME and saline. Tumors formed in Alg-HA were the largest on average of all the gel groups. Columns represent a single tumor, and rows represent a single magnification. 2× magnification the scale bars represent 2 mm. 4× magnification the scale bars represent 1 mm. 10× magnification the scale bars represent 400 micrometers.

FIG. 16 shows according to an exemplary embodiment of the invention % Necrosis as determined by a blinded pathologist for each tumor in all experimental groups.

FIG. 17 shows according to an exemplary embodiment of the invention representative CD31 staining from each group. No significant differences were observed by a blinded pathologist. Background melanin made clear analysis challenging. Scale bar represents 250 micrometers.

FIGS. 18A-B show according to an exemplary embodiment of the invention in FIG. 18A number of lymphocytes per tumor found in 10 high power fields (40× magnification) for all experimental groups. FIG. 18B, representative high-power images of CD3 stained tumors where lymphocytes are stained dark brown. Scale bar represents 50 micrometers.

### DETAILED DESCRIPTION

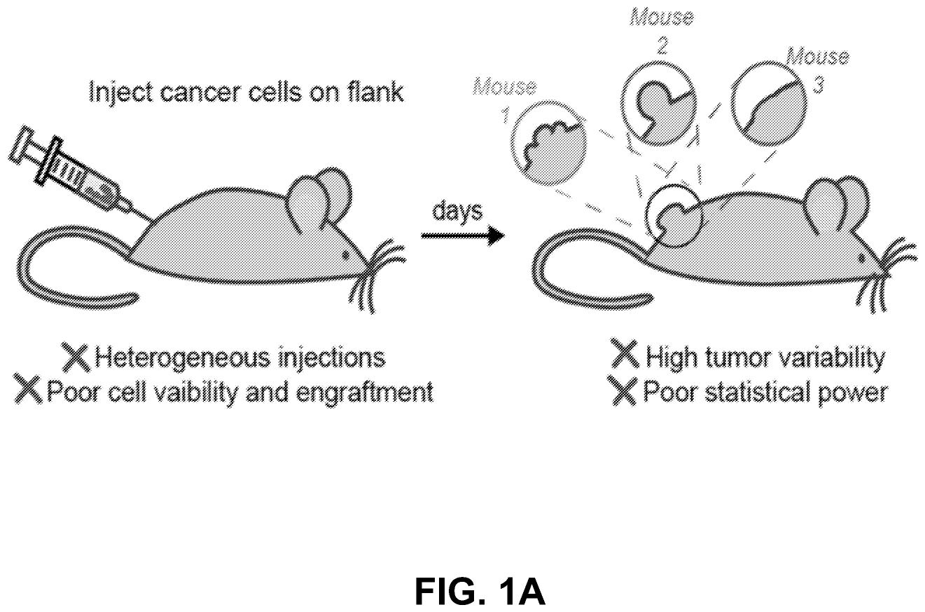

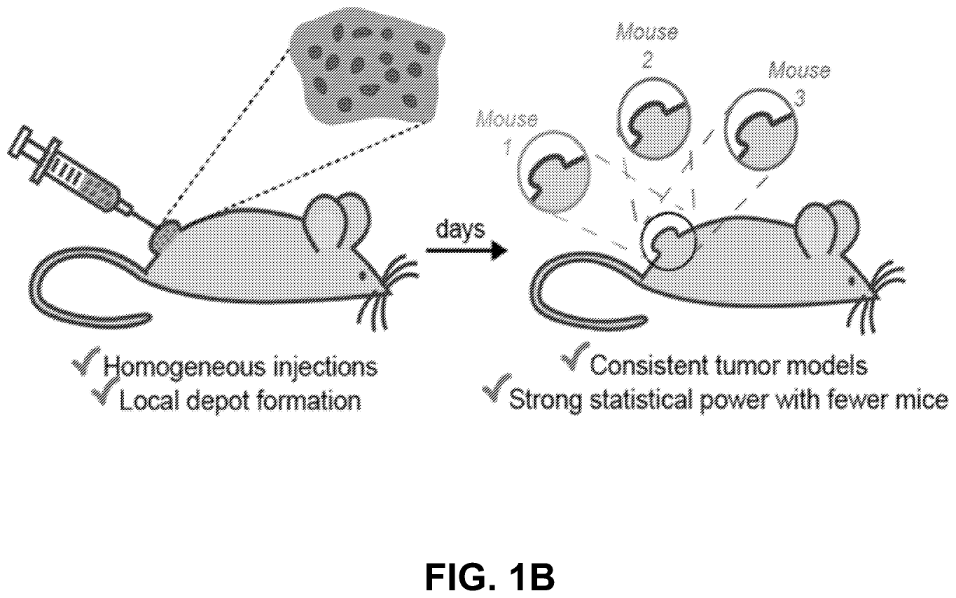

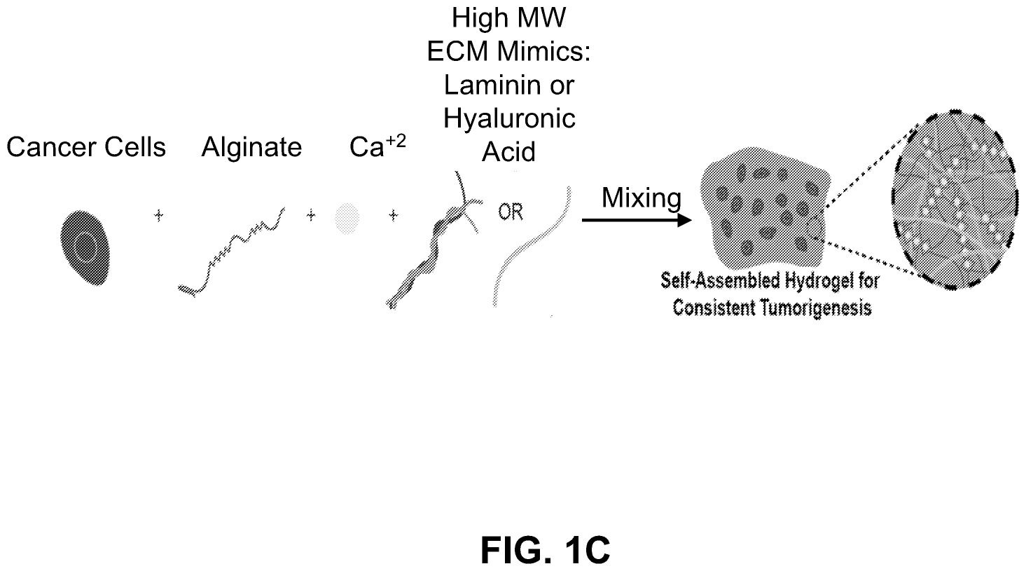

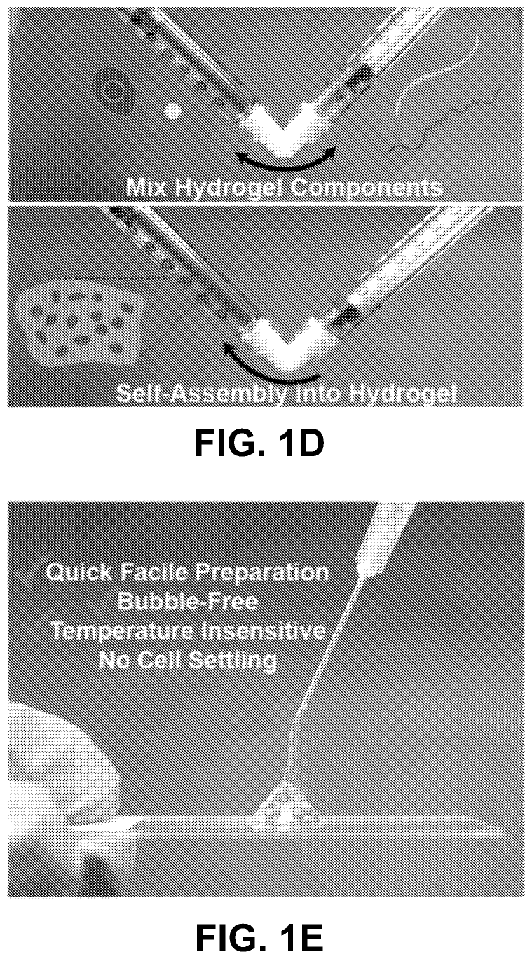

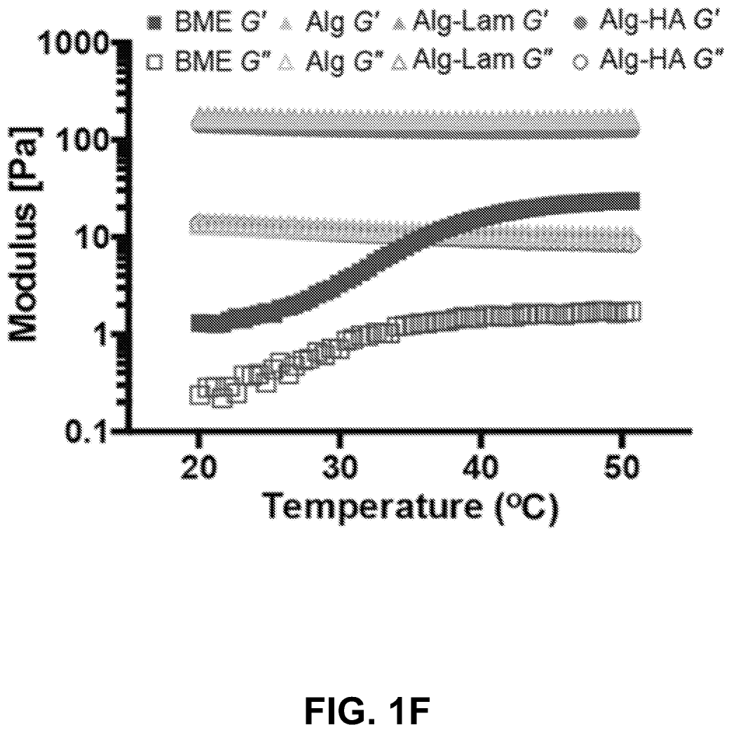

Methods of embodiments of the invention have been validated with the B16F10 melanoma model (FIG. 1B). Results of exemplary embodiments will be discussed first followed by methods used to achieve these results.

### Results

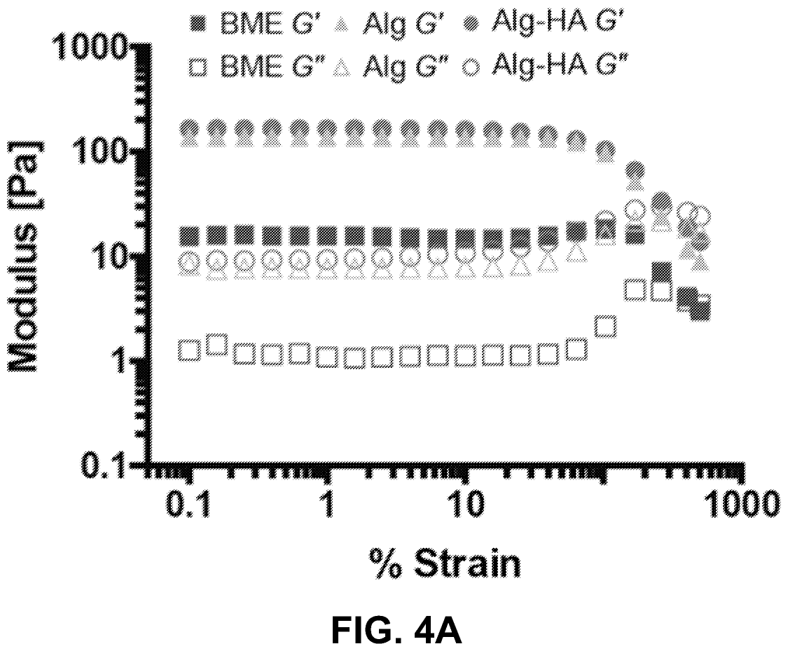

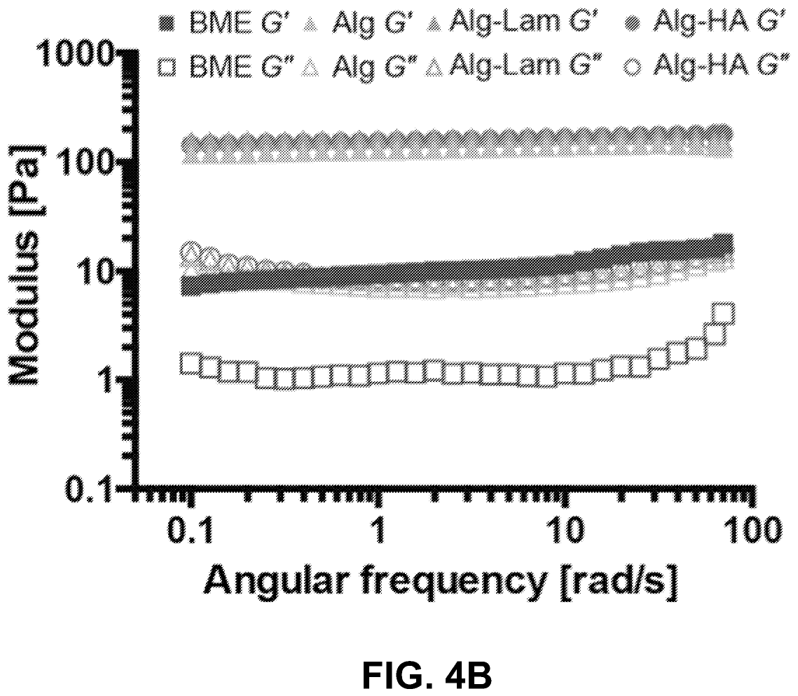

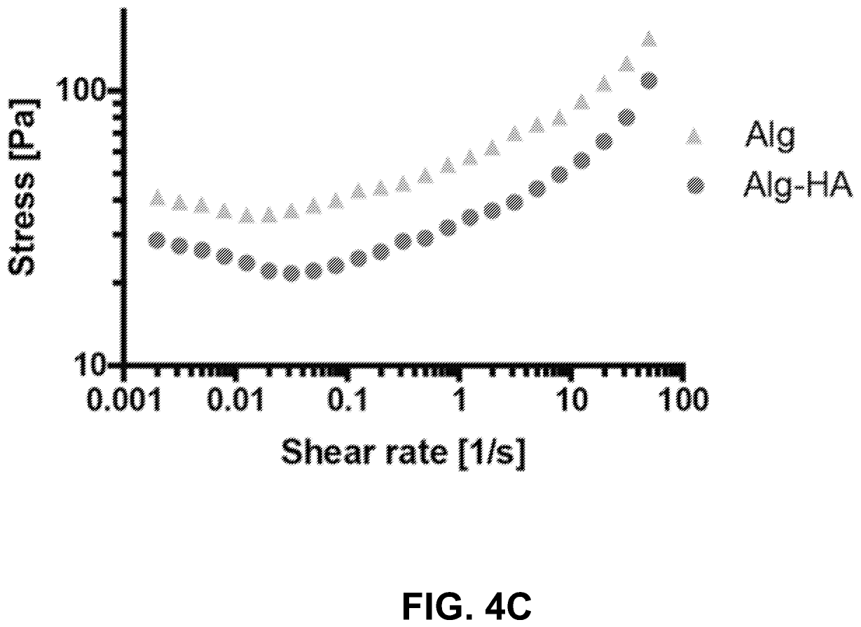

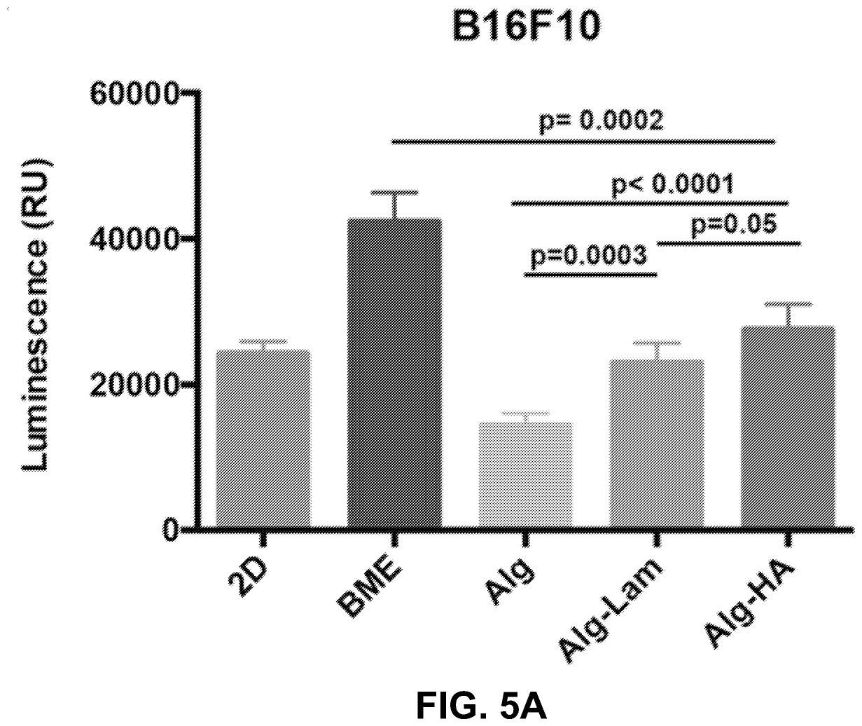

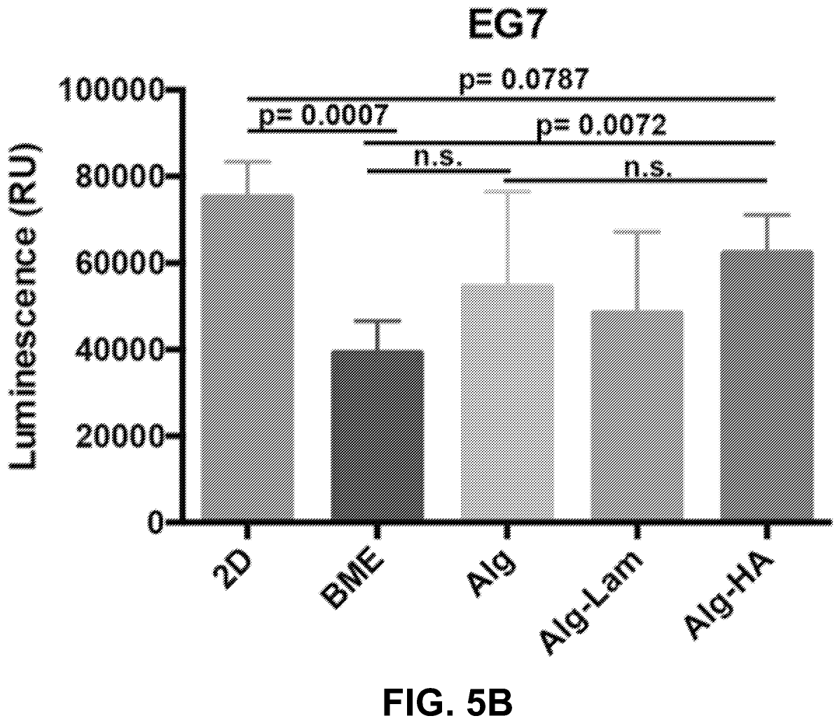

In an exemplary embodiment, alginate-based hydrogels were used due to great biocompatibility, and their mild and rapid formation by mixing with calcium (FIG. 1C). This facile fabrication method does not involve complex chemical reactions affording efficient encapsulation of viable cancer cells. These cell-encapsulated injectable hydrogels quickly self-assemble with 30 seconds of mild mixing of two precursor solutions (e.g., alginate (Alg) and calcium) in a dual-syringe setup that results in the hydrogels pre-loaded in a syringe and ready for injection (FIGS. 1D-E). Further, these alginate-based hydrogel formulations can be easily supplemented with laminin (Alg-Lam) and hyaluronic acid (Alg-HA), which are components of the ECM in endogenous tumors20,21. All alginate-based hydrogel formulations display temperature-insensitive dynamic mechanical properties (FIG. 1F and FIGS. 4A-C). In contrast, BME exhibits irreversible temperature-dependent gelation and much weaker dynamic mechanical properties. For the studies pertaining to the invention, laminin and HA were added in the maximum quantities possible to formulate consistent alginate-based hydrogels and maintain injectability. Short-term cell viability studies in two commonly used cancer cell lines indicate that the hydrogel formulations are not cytotoxic and that the addition of adhesion motifs such as laminin and HA improve cell growth (FIGS. 5A-B). Different cell lines interestingly demonstrated variable responses to growing in BME.

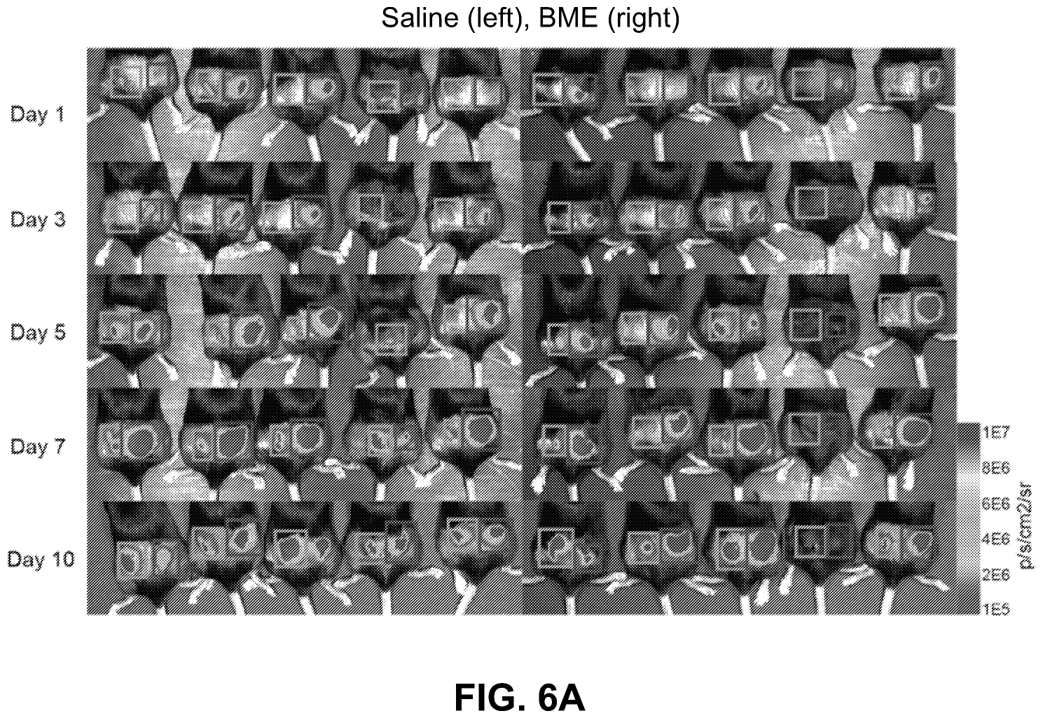

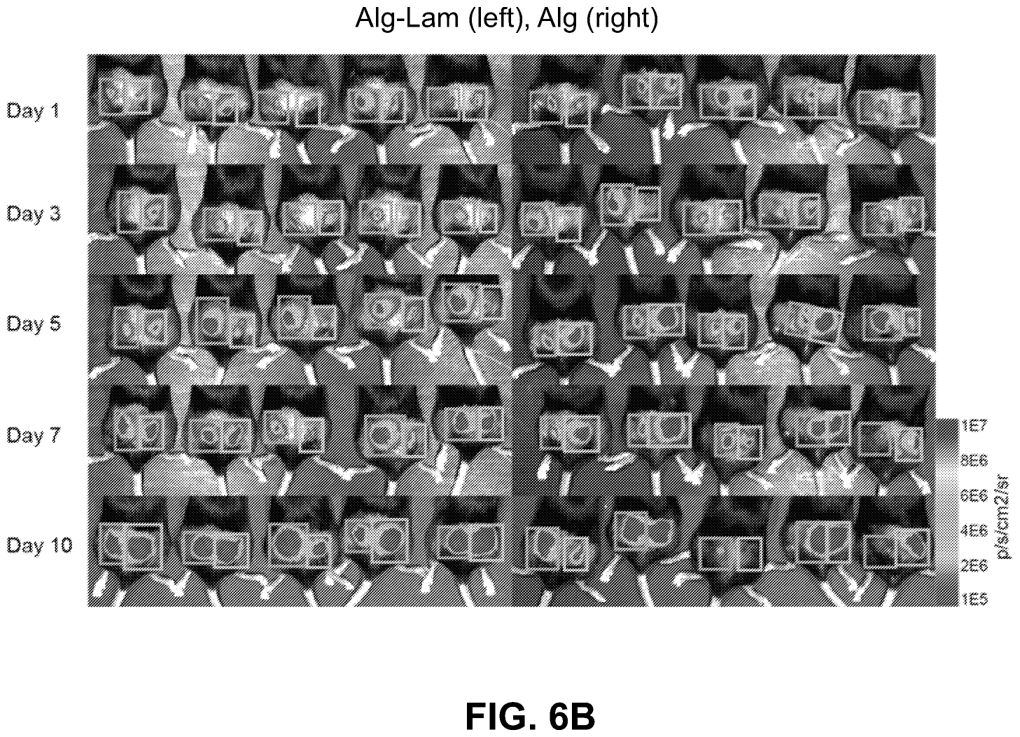

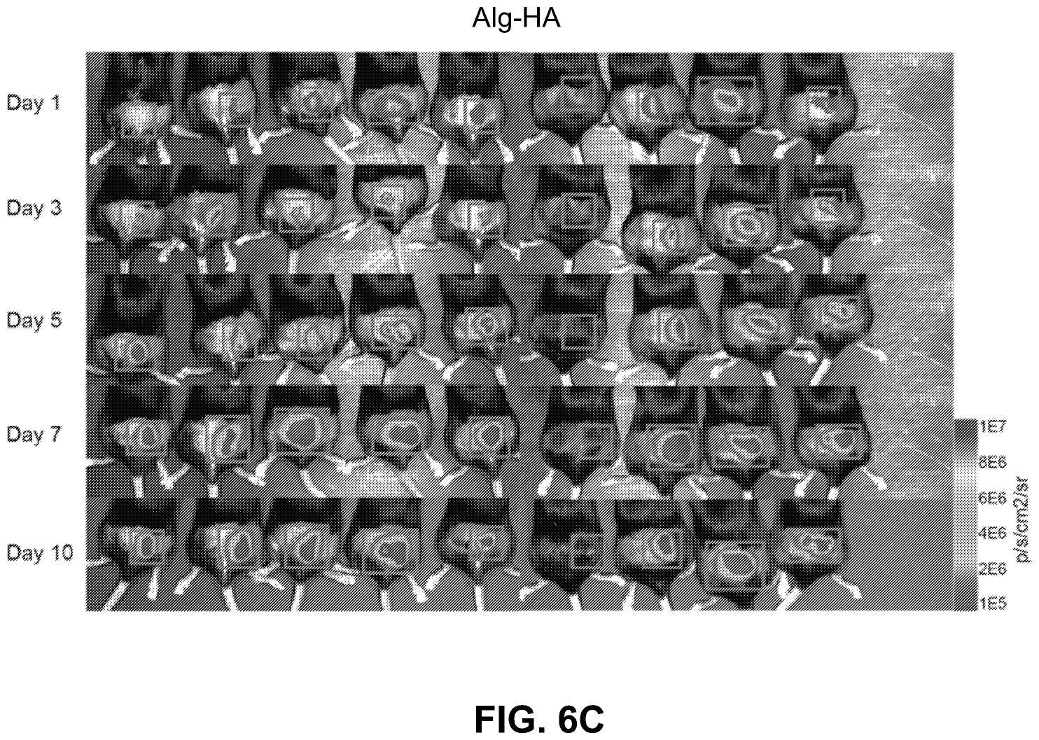

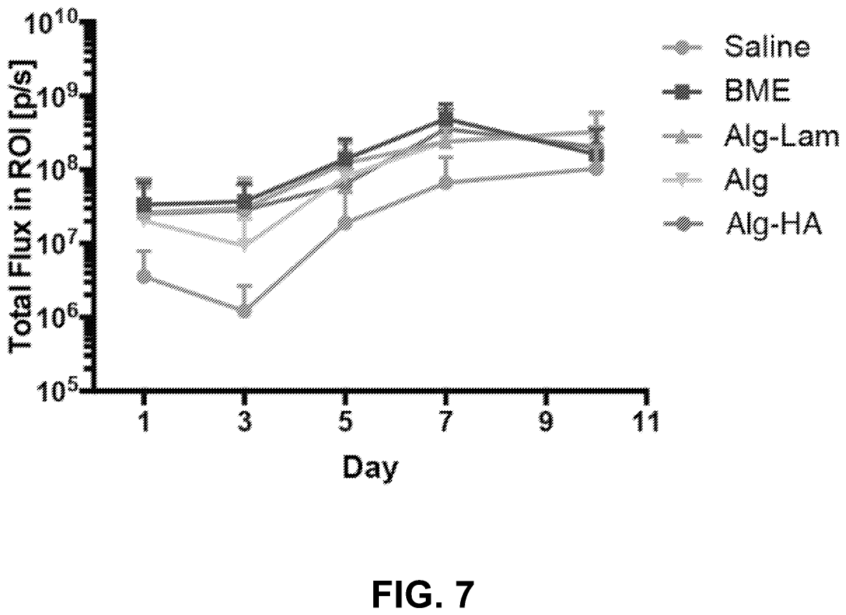

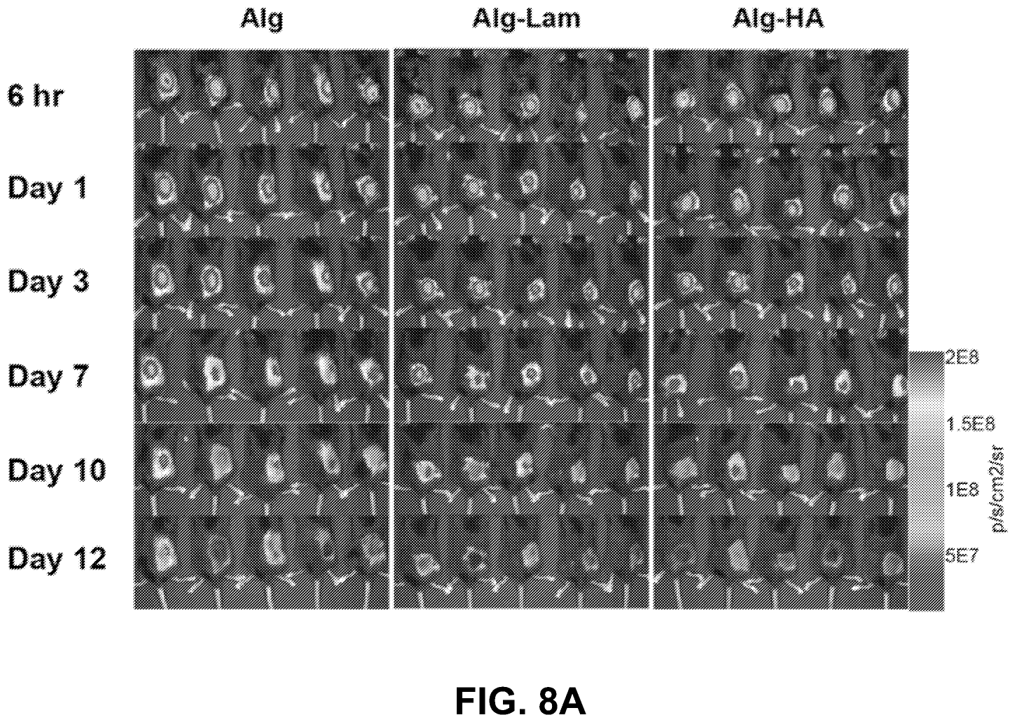

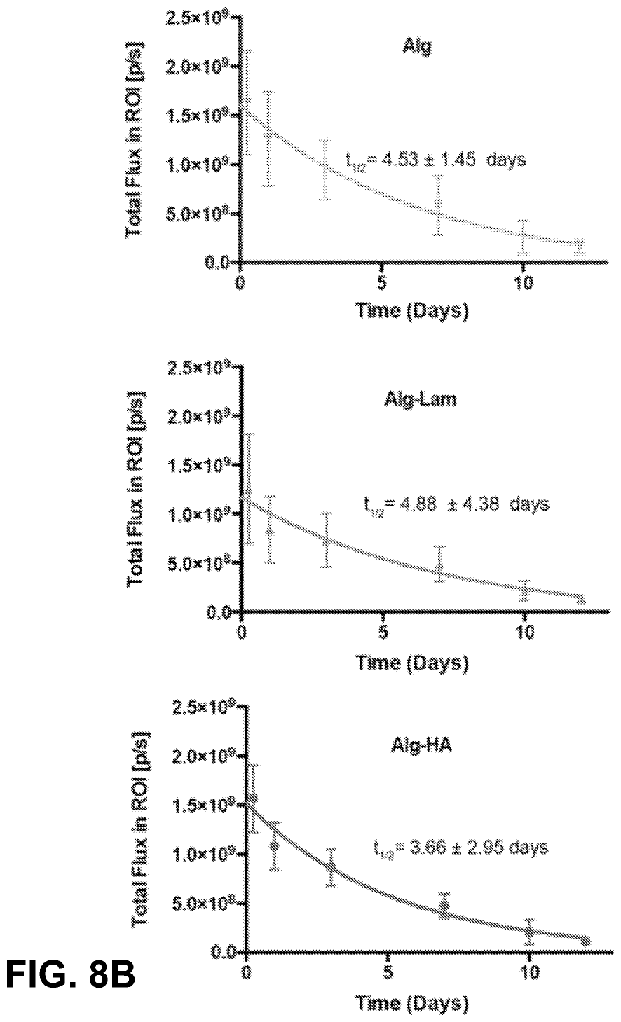

To demonstrate the method, Luc+B16F10 cancer cells were encapsulated in 50 μL of hydrogel formulations and injected subcutaneously on the flank of C57BL/6 mice through a 21 G needle. Tumor growth was compared with tumors administered in 50 μL of BME and saline. Tumor formation was observed for the first 10 days with in vivo imaging (FIGS. 6A-C and FIG. 7). Tumors established with saline displayed lower signal on Day 1 than all other formulations, indicating poor cell viability and retention after inoculation, corroborating observations of inconsistent tumor formation. These initial tumor growth data were additionally fit to a Gompertz model. The fits suggest that the hydrogel approach does not hinder proliferation and may improve end carrying capacity of the tumor compared to saline controls. Degradation of alginate was also monitored using in vivo imaging and fluorescently labeled alginate. Results demonstrated that alginate degraded over the course of 10-12 days with a half-life of approximately 4 days (FIGS. 8A-B). This time-scale of degradation aligns with the B16F10 cells growing into a robust tumor, remodeling the matrix, and degrading the alginate, suggesting there should not be significant effects of alginate being present during treatment.

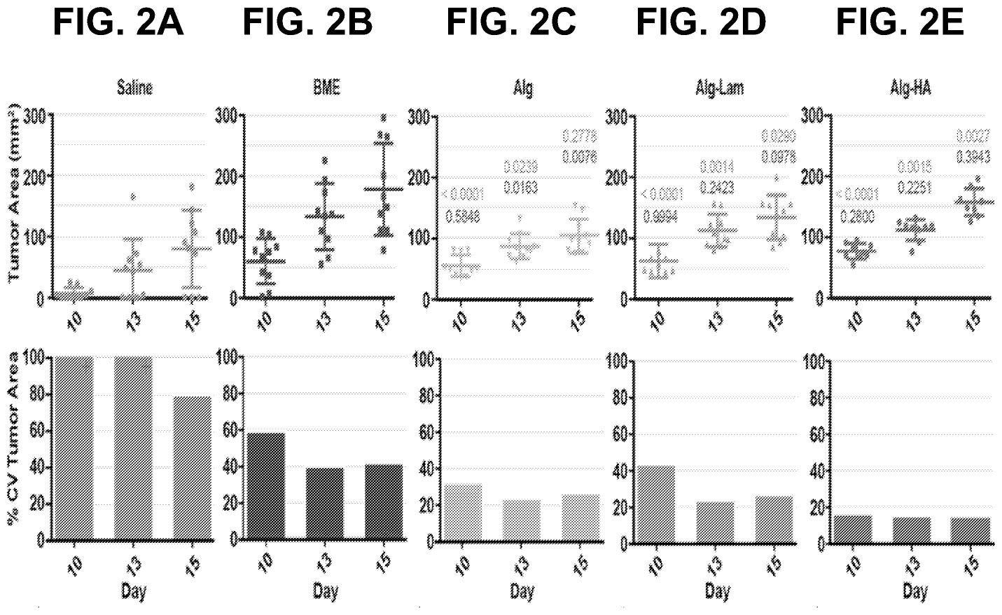

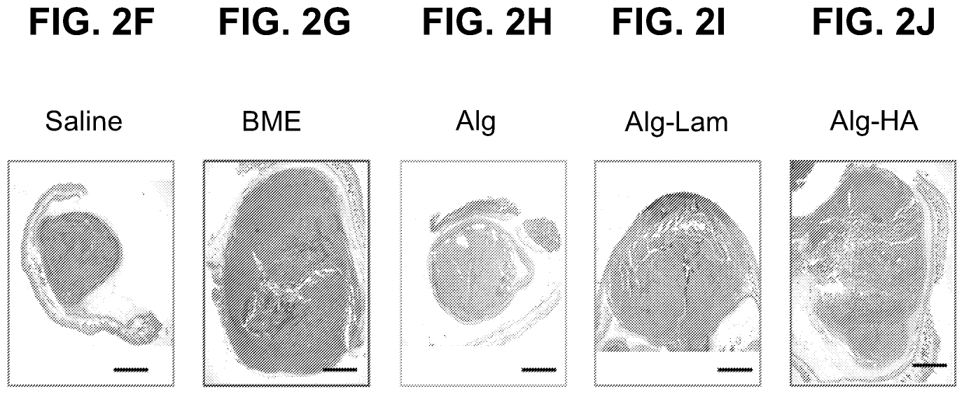

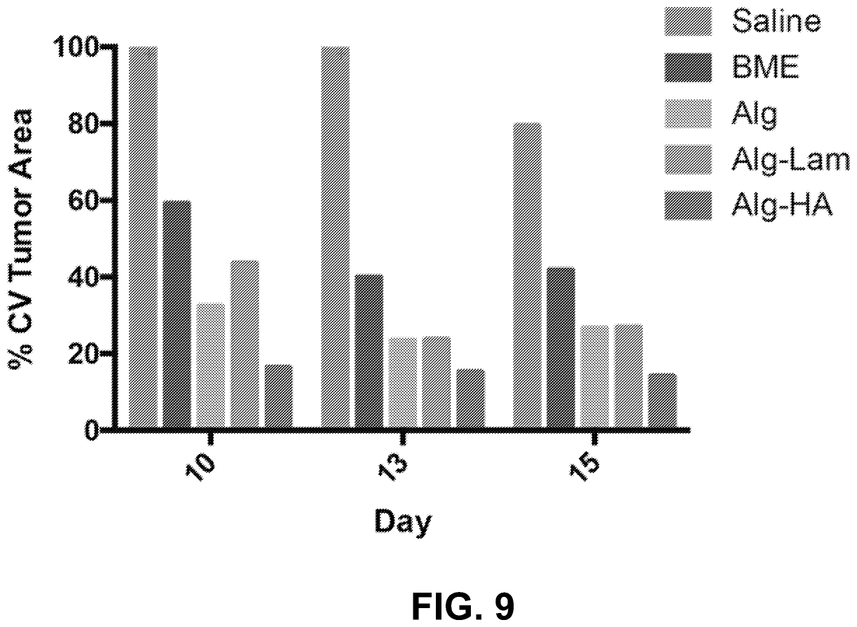

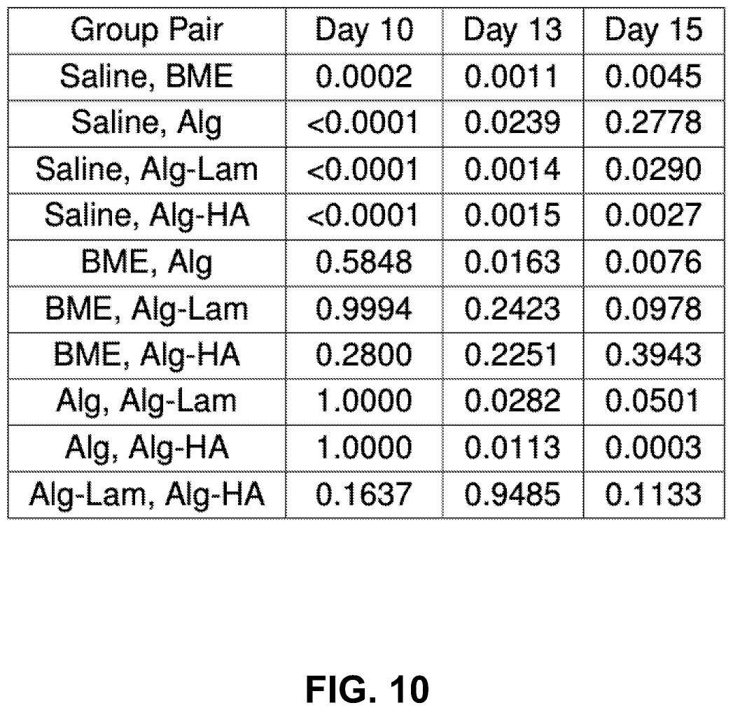

Once tumors reached 100 mm², tumor area was measured over time with digital calipers (FIGS. 2A-E, FIG. 9 and FIG. 10). The coefficient of variance (% CV) was quantified (FIGS. 2A-E) and revealed that tumors inoculated in a saline vehicle showed the greatest variance among all of the groups evaluated. Tumors formed in BME showed the fastest growth, but also demonstrated a high % CV (40%). Alginate hydrogel alone (Alg) yielded moderate tumor growth with lower % CV (<25%), while the hydrogels comprising tumorigenic ECM components Laminin (Alg-Lam) and HA (Alg-HA) exhibited enhanced tumor growth while maintaining low % CV values (<25% and 15%, respectively) that were less than half that of tumors from saline and BME. The Alg group likely showed the slowest group due to the absence of an additive stimulating tumorigenic biomolecule, like laminin or HA.

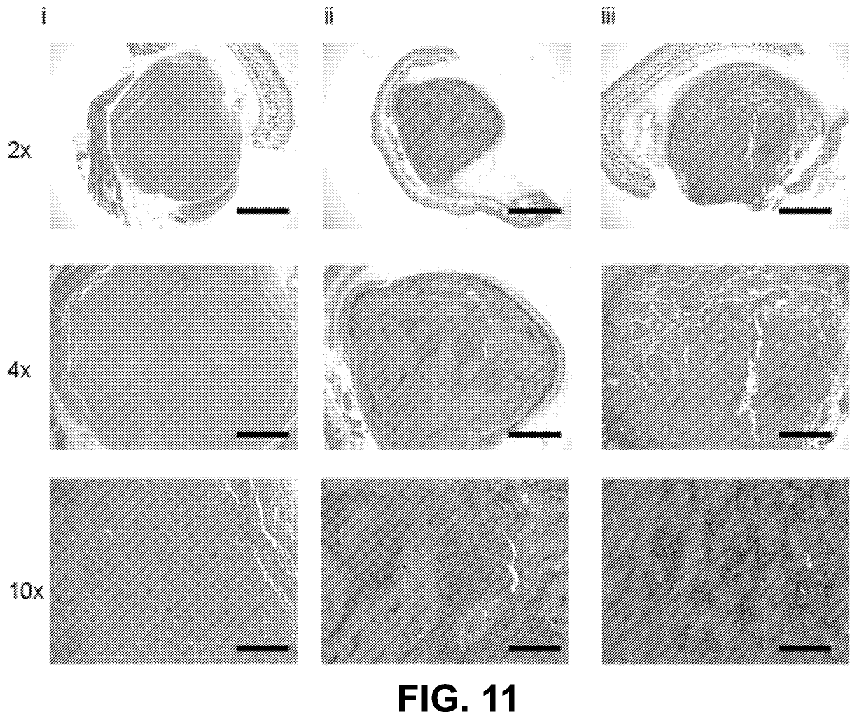

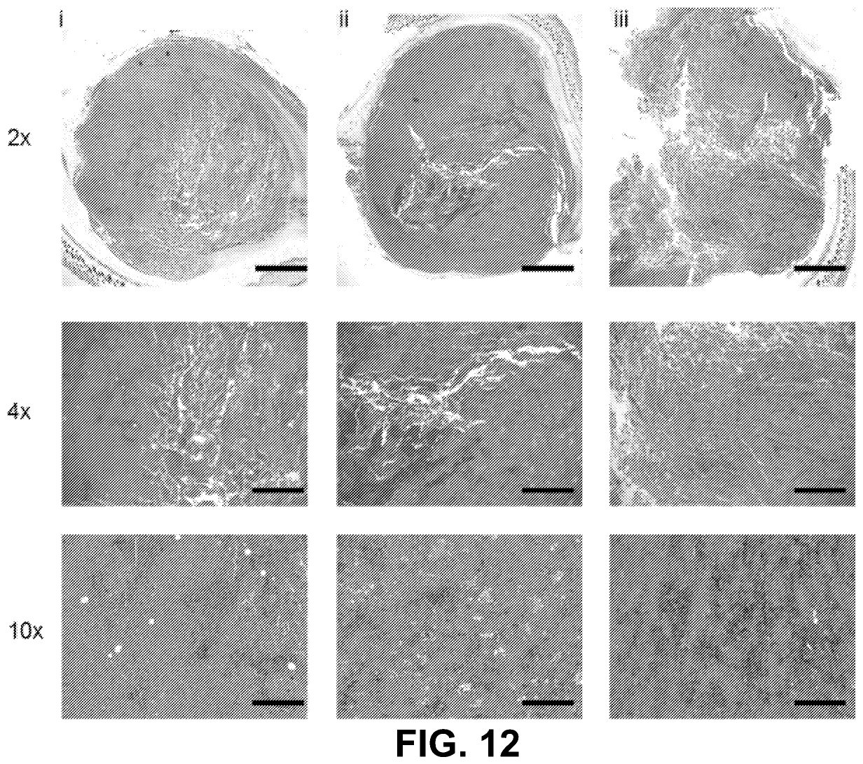

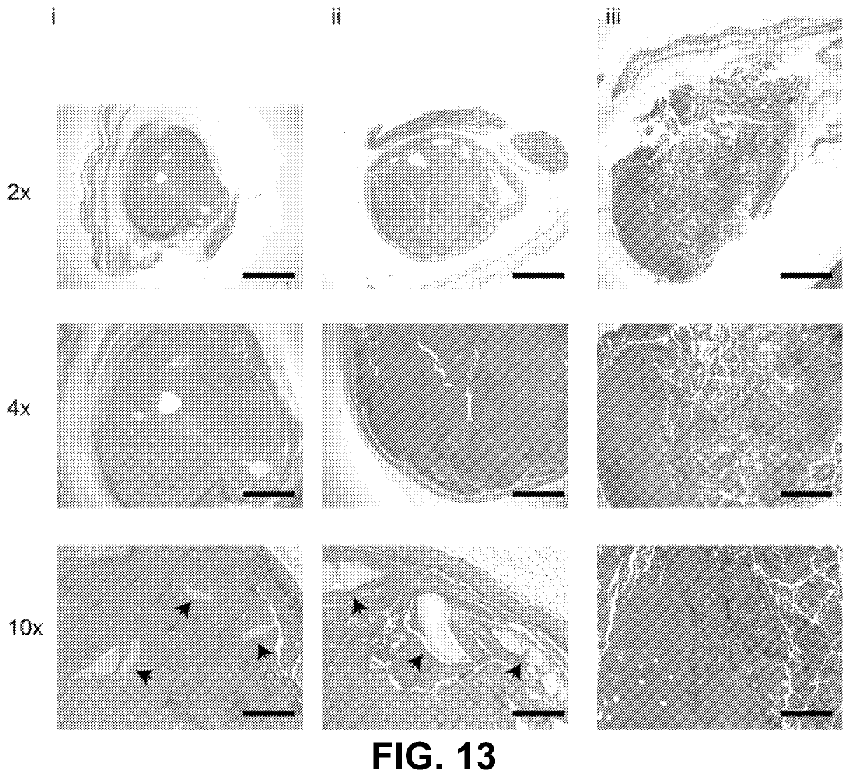

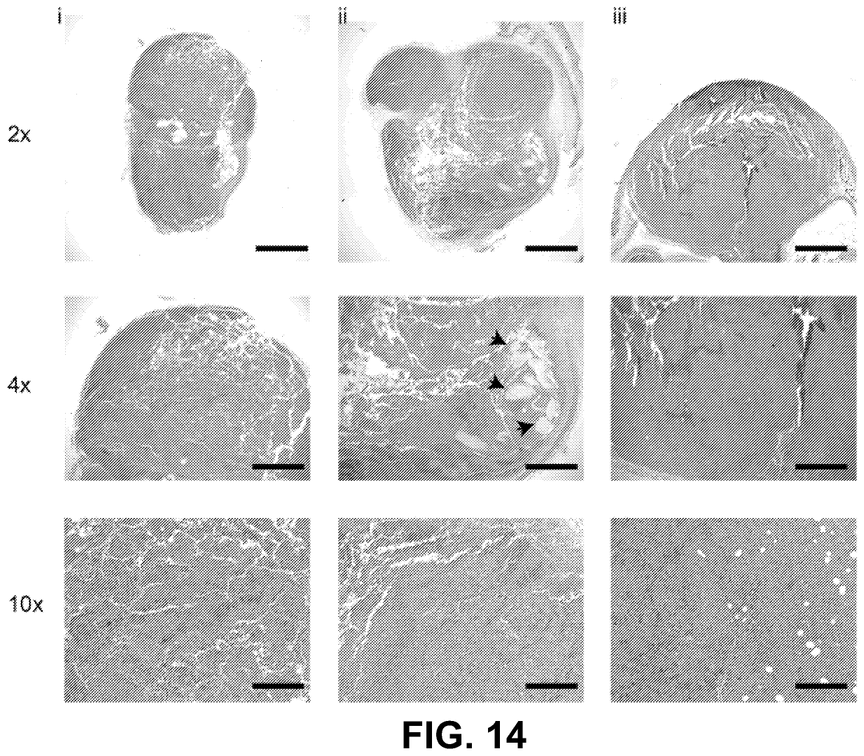

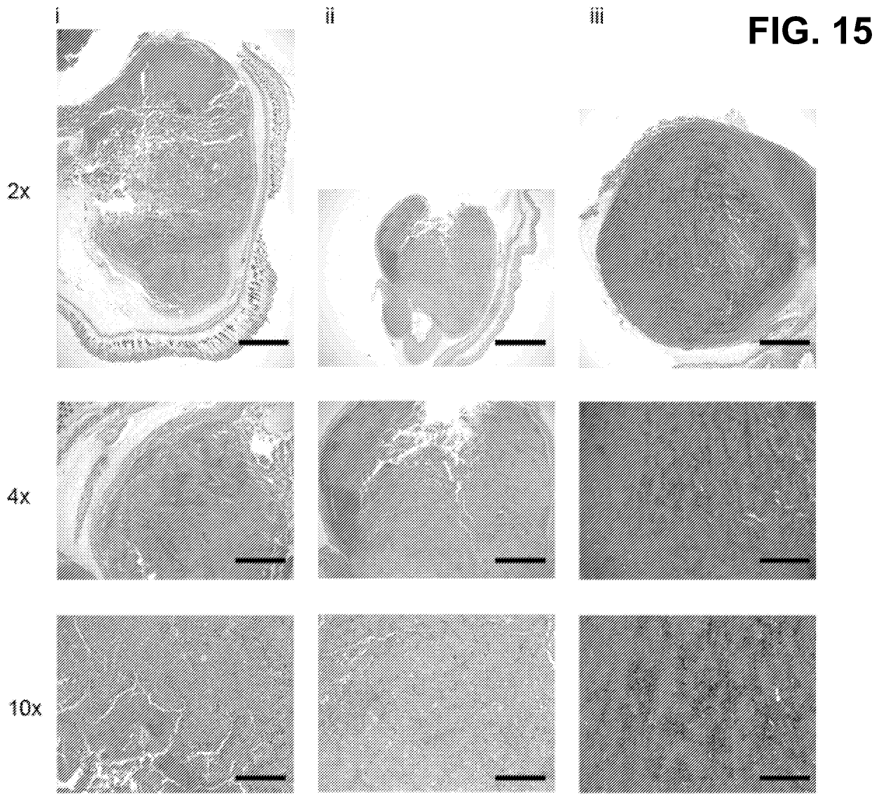

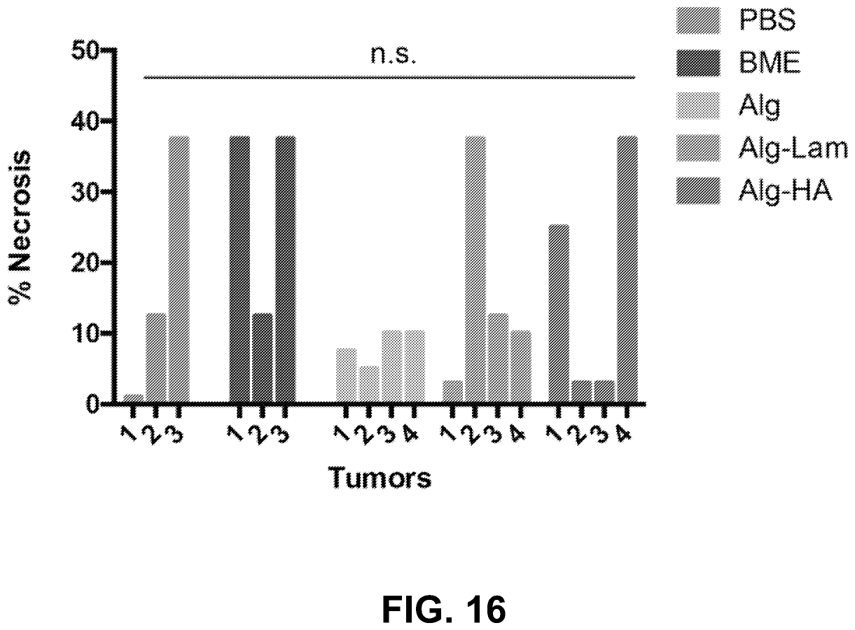

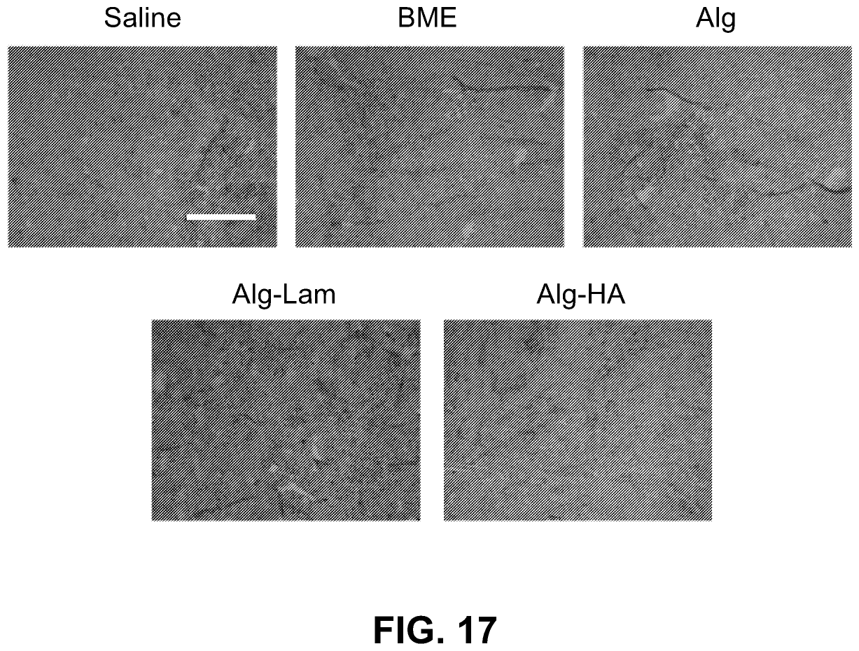

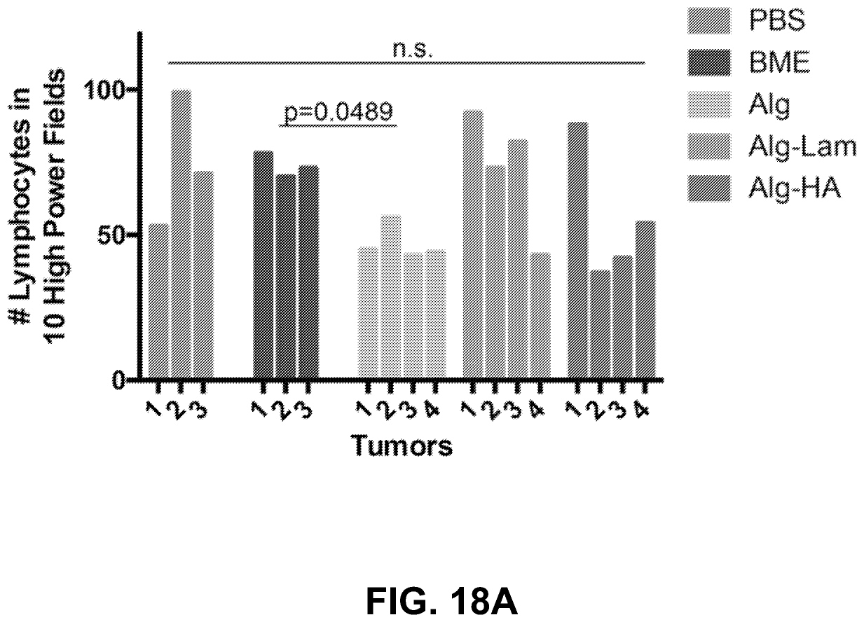

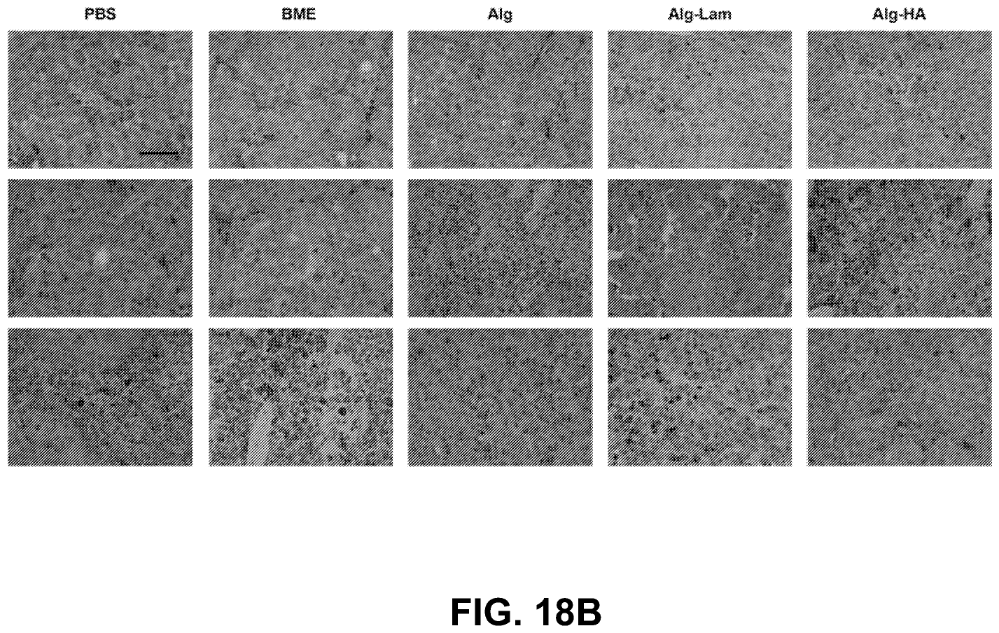

Tumor histomorphology on day 15 post-inoculation evaluated by a blinded pathologist was comparable for tumors formed in hydrogel groups (regardless of composition), BME, and saline (FIGS. 2F-J), suggesting that the invented delivery method does not change long-term tumor morphology and applicability of tumors formed in hydrogels for preclinical studies. Replicate representative images suggest the delivery method does not change the resulting tumor histomorphology (FIGS. 11-15). Percent necrosis analysis in each tumor and staining with CD31 by a blinded pathologist also suggested comparable angiogenesis and blood supply in all groups (FIGS. 16-17). Finally, an analysis of lymphocyte infiltration revealed similar numbers of lymphocytes infiltrating the tumors in all groups, except the Alg hydrogel group showed slightly reduced infiltration (FIGS. 18A-B).

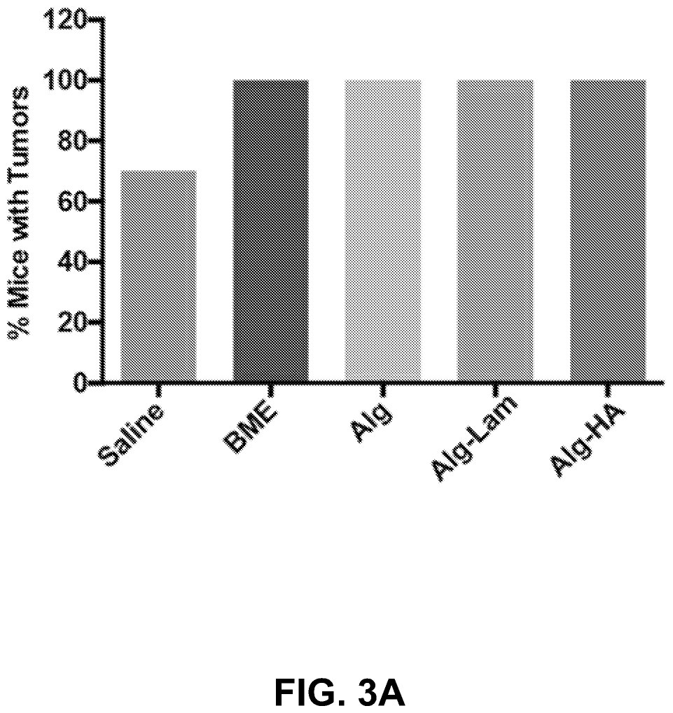

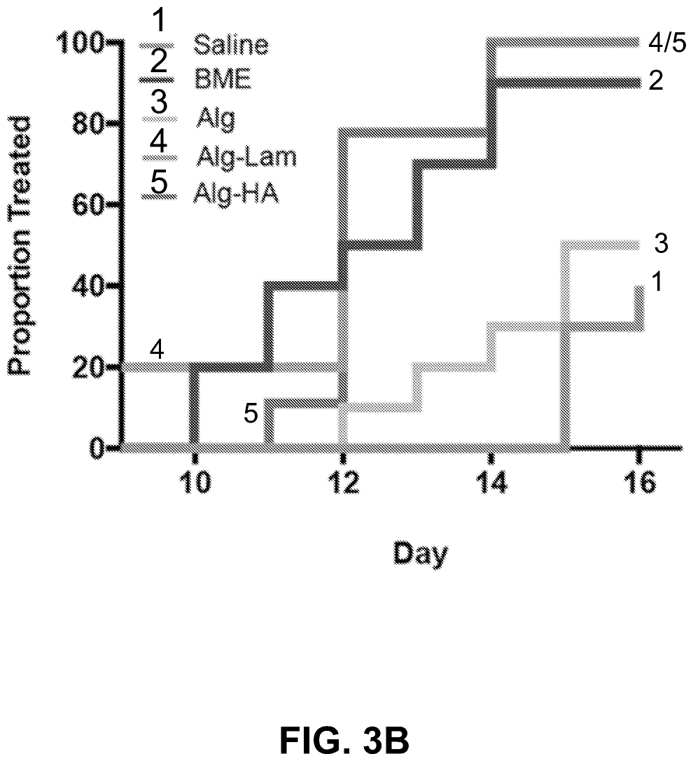

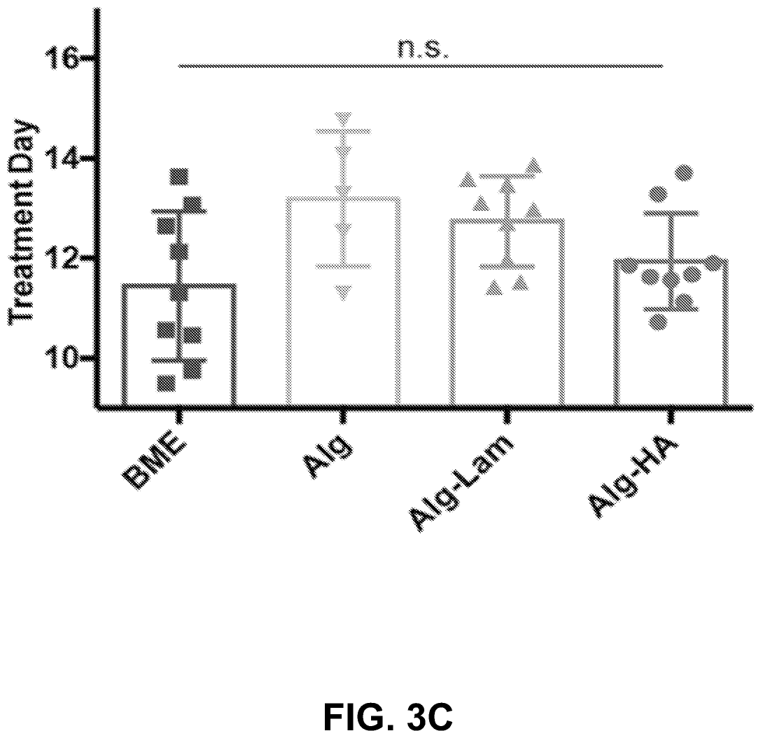

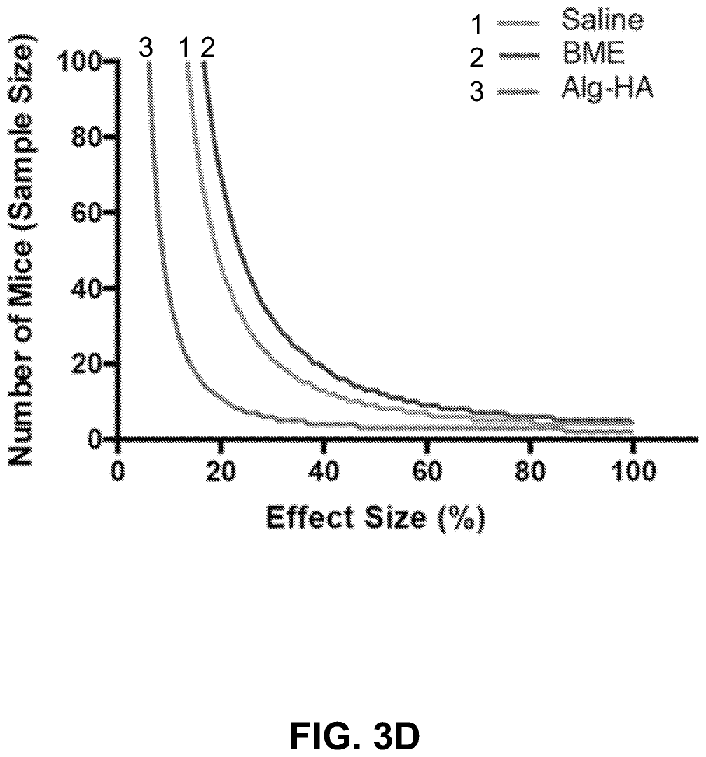

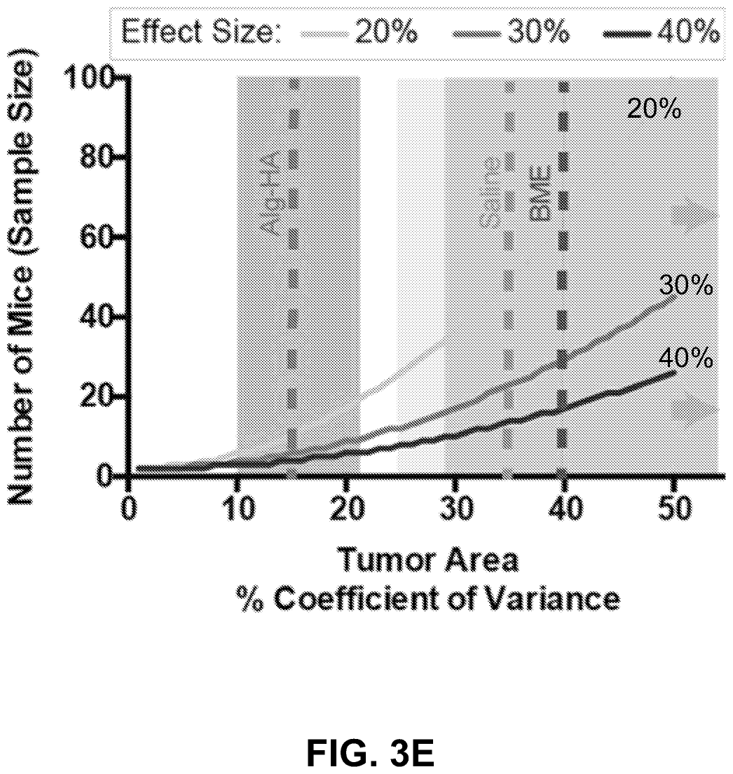

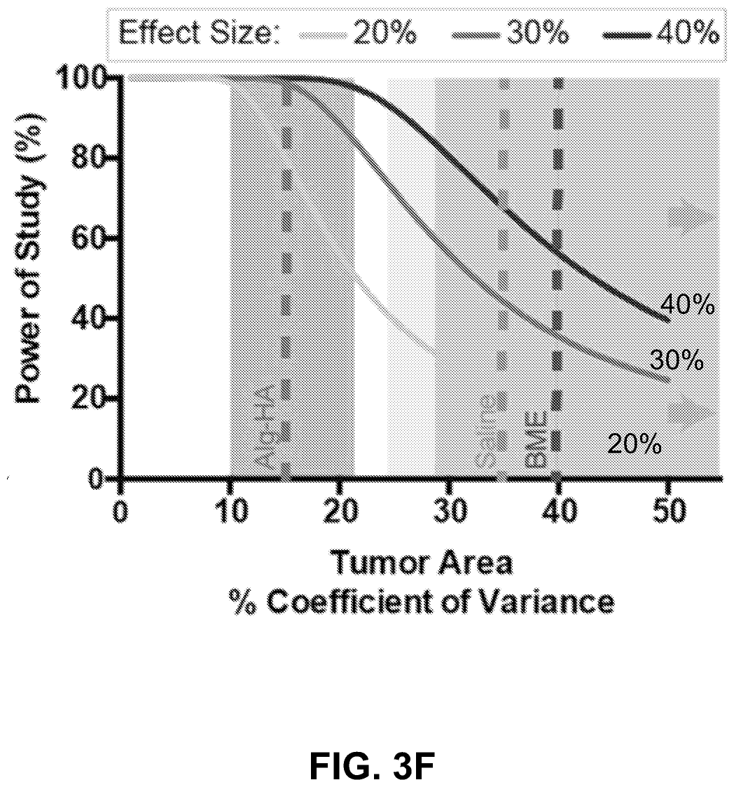

Researchers aim to start treatment when tumors are a given size (e.g., 100 mm²), which for practical reasons would preferably occur on the same day for all mice; however, treatment must be staggered if tumors grow at different rates. Indeed, 30% of the mice inoculated with cells in saline did not form tumors during the 15-day study (FIG. 3A), corroborating previously reports. The inventors therefore analyzed the effect of tumor variance on treatment day by identifying the proportion of mice treated in each group over time using interpolation to predict the day each mouse could be treated (FIG. 3B). The average treatment day and corresponding standard deviation for each formulation were also determined (FIG. 3C), corroborating the variance in tumor growth described above. Tumors inoculated with BME exhibited high variability for time-to-treatment (11.5±1.5 days), highlighting that researchers would have to stagger experimental treatments continuously over the course of 3-4 days. In contrast, tumors inoculated in Alg-Lam and Alg-HA hydrogels exhibited lower variability (12.7±0.9 and 11.9±1.0 day, respectively), resulting in a more convenient treatment schedule and likely improving the precision of treatments.

Reduced model variance reduces type II error, allowing researchers to use fewer animals and observe differences between treatments with higher power. Assuming that the variance in tumor areas due to the model at the start of treatment is representative of the variance throughout treatment, it is possible to determine the impact of the improved model on design of sufficiently powered studies (FIGS. 3D-F). The reduction in the tumor variance observed with Alg-HA hydrogels compared to saline and BME results in a dramatic reduction in the number of mice required to observe differences between treatments. Calculations were performed with only best performing the Alg-HA hydrogel group and control groups to reduce the number of groups and complexity in this power analysis. For example, to observe a 30% difference between means of two treatment groups with 80% power, an Alg-HA-based B16F10 model would require approximately 80% fewer mice than a BME-based model (FIG. 3D). A reduction in model variance therefore reduces the number of mice required for sufficiently powered preclinical cancer studies. Alternatively, if researchers plan to use 10 mice in each experimental group (a typical group size), an Alg-HA-based model would enable observation of significant differences between groups down to effect sizes of only 21%, whereas a BME-based model would only permit observations of effect sizes larger than 56% (FIG. 3E). The reduced variance of alginate-based tumor models can therefore enable the observation of more subtle effects and may lead researchers to important discoveries that would otherwise be missed. If researchers plan to use 10 mice per group, a decrease in model variance yields much higher-powered studies. In a study evaluating a 30% effect size in 10 mice, a Alg-HA-based model enables a 60% increase in power over a BME-based model (FIG. 3F).

The method according to this invention has the capacity to enhance the reliability of tumor formation and consistency of tumor growth for the widely used B16F10 model, demonstrating an opportunity to improve preclinical cancer models to aid observation, increase study power, and reduce resource usage (i.e., fewer mice and less researcher time). This approach has great potential to expand to most other subcutaneously administered tumor models, such as, but not limited to, 4T1, MC38, and LLC1 models. The hydrogel encapsulation procedure demonstrated herein, which only requires a simple mixing step using commercially available precursors and standard supplies, is accessible to all preclinical cancer researchers. The inventors showed improved results over current methods that utilize either saline or BME. While extensive research has focused on developing biomaterials for tissue engineering applications, the use of biomaterials to generate more reproducible in vivo cancer models has not been extensively explored. We demonstrate here, using predictive modeling, that a biomaterials-enhanced cancer model can reduce technical burden and simplify study design.

### Methods

Methods described are exemplary methods leading to the results described above. A skilled artisan appreciates that certain variations could be made without departing from the scope of the invention. Hence the specific examples should not be regarded as limiting to the scope of the invention.

### Hydrogel Formation by Self-Assembly

Hydrogels were prepared using a dual syringe mixing technique. Briefly, a stock solution of sterile Alginate (Pronova UP LVG) (5 wt %) was prepared by adding buffered saline to the polymers and allowing them to dissolve over 1 day at 4 degrees Celsius. This alginate is higher in G content, leading to reduced immunogenicity. A stock solution of alginate (5 wt %) and HA (Lifecore Biomedical, 1.5 MDA, 1.25 wt %) was also prepared in saline. Biopolymer stock solutions were loaded into one syringe at the volume needed to reach the desired final concentration (1 wt % polymer total, and 10 mM calcium). A stock solution of calcium sulfate (250 mM) was prepared in water in a large container in the form of a slurry. The calcium sulfate stock solution was stirred vigorously and quickly added to an eppendorf tube containing cells and saline. Laminin Mouse Protein (Thermo Fisher) was added to the solution containing calcium and cells to a final concentration of 1 mg/mL. HA was added to a final concentration of 20 mg/mL. The stock solution of calcium and cells was then moved to a luer lock 1 mL syringe. The syringes containing the two stock solutions were then connected using a female-female luer lock mixer and the gels were prepared by mixing for 30 pumps. Once mixed, the cell-loaded hydrogels were pushed into one of the syringes, which was then removed from the leur lock mixer and equipped with a needle for application. Alternatively, the calcium sulfate can be premixed with the alginate prior to encapsulating cells in the case that cells are sensitive to direct contact with calcium.

### Conjugation of Cyanine7 Fluorescent Dye to Alginate

Fluorescent alginate was prepared using carbodiimide chemistry according to established protocols, whereby Sulfo-Cyanine7 amine (5 mg, 0.0062 mmol; Lumiprobe) was dissolved under stirring in 15 mL of an alginate solution (10 mg/mL) formed in 0.1 M MES buffer at pH 6 (Thermo Fisher). Sulfo-NHS (41 mg, 0.19 mmol; Biovision) and 1-ethyl-3-(3-dimethylaminopropyl)carbodiimide (EDC; 72.5 mg, 0.38 mmol; Sigma-Aldrich) were successively added, and the reaction mixture was stirred at room temperature for 20 hours. The crude product was dialyzed against deionized water for 3 days (3.5 kDa MWCO) and lyophilized until dried.

### Rheological Characterization

Rheological testing was performed using a 20 mm diameter serrated parallel plate at a 750 μm gap on a stress-controlled TA Instruments DHR-2 rheometer. All experiments were performed at 37 degrees Celsius to be representative of physiological conditions. Frequency sweeps were performed at a strain of 1%. Temperature sweeps were performed at a strain of 1% and a frequency of 10 rad/s. Amplitude sweeps were performed at frequency of 10 rad/s. Independent hydrogel formulations were mixed for each test.

### B16F10 Cell Culture

Luc+B16F10 cells were purchased from ATCC (ATCC CRL-6475-LUC2). They were cultured in Dulbecco's modified Eagle's media containing 10% FBS, 1% penicillin-streptomycin, and, if Luc+, with 10 μg/mL Blasticidin. Cells were split at a 1:5 ratio every 3 days when approximately 80% confluent. For in vivo experiments, they were injected at passage 3 when 50% confluent. Cells tested negative for mycoplasma using Lonza Mycolert Mycoplasma Detection Kit prior to experiments.

### EG7 Cell Culture

EG7 cells were purchased from ATCC. They were cultured in RPMI media containing 10% FBS, 1% penicillin-streptomycin, and 0.05 mM 2-mercaptoethanol. Cell media was supplemented every 2 days and upon confluency (1e6 cells/mL), cells were split by dilution to a seeding density of 100,000 cells/mL. Cells tested negative for mycoplasma using Lonza Mycolert Mycoplasma Detection Kit prior to experiments.

### Short-Term Viability Assay

Promega CellTiter-Glo 3D Cell Viability Assay was used to characterize the short-term cell viability in different formulation conditions. Cells were seeded between 5000 and 10,000 cells per well in an opaque 96-well plate in 100 μL of media or gel per well. Relative viability was measured after 1 day in culture by adding 100 μL per well of the CellTiter-Glo reagent, mixing for 5 minutes, allowing the plate to sit for 25 min, and then reading the luminescent signal with a 1 second integration time.

### In Vivo Tumor Growth Experimental Setup

Luc+ B16F10 cells (Passage 3) were administered to each mouse subcutaneously on the flank in a volume of 50 μL volume containing 400,000 cells using a 1 mL luer-lock syringe with a 21 G needle. In all, 400,000 cells were chosen as an intermediate number of cells based on previously reported protocols. Immunocompetent female C57BL/6 mice from Charles River Laboratory (6-8 weeks) were used for all experiments. Each group contained 9 or 10 replicates (n=10 for saline, BME, alginate and Alg-Lam; n=9 for Alg-HA). Experimental groups were blinded and randomized by cage. Hydrogel formulations comprising encapsulated cells were prepared in syringes in the laboratory 30 min prior to inoculation. Cell suspensions were prepared in saline or BME and kept on ice prior to loading into syringes immediately before inoculation. Double the amount of saline volume was prepared to follow established procedures to prevent cell aggregation in the syringe.

### Murine Tumor Growth Experiment

For the first 10 days of in vivo tumor growth experiments, an In Vivo Imaging System (Lago) was used to monitor tumor progression. Firefly luciferin was delivered subcutaneously (150 mg/kg mouse body weight) in 200 μL injection volumes. Luciferase has been found to have immunogenic effects in certain models and was equally used for imaging in all groups for experiments, so side-effects should be uniform among groups. After waiting 10 min, images were recorded with an exposure of 30 seconds every 5 minutes for a period of 15 minutes until maximum flux was reached. Total flux of photons in the tumor region of interest (ROI) was used to quantify tumor growth. Aura imaging software was used to collect and analyze data. Starting on day 10 following tumor inoculation, tumors were measured on each mouse using digital calipers. An area was calculated using length and width caliper measurements. The percent coefficient of variance was calculated as the standard deviation divided by the mean.

### In Vivo Alginate Degradation

Hydrogels were formulated as previously described though comprising 0.2 wt % Cy7-labeled alginate and 0.8 wt % plain alginate. B16F10 cells were encapsulated and injected in the hydrogels. Alginate degradation was monitored using an in vivo imaging system and quantified as the total photons in the ROI surrounding the tumor. An exposure of 1 second was used to collect images with an excitation/emission of 720/790 nm.

### Treatment Day Analysis

The treatment day was calculated for each mouse by interpolating between two timepoints to when the tumor area reached 100 mm². Interpolation was performed assuming both exponential and linear tumor growth. The average treatment day and standard deviation for each group were calculated from the individual mice. Mice that required extrapolation before day 9 and after day 16 (tumor reached 100 mm² before measurement began or after measurements stopped) were removed from the analysis. The following equations describe the linear interpolation approach, where t₁₀₀ represents the day when the tumor reaches 100 mm², A₁ and A₂ represent area measurements for corresponding days t₁ and t₂,

$${t}_{100} = \frac{ln ( 100 ) - b}{a} where$$

$$a = \frac{ln ( {A}_{2} ) - ln ( {A}_{1} )}{{t}_{2} - {t}_{1}} and$$

$$b = ln ( {A}_{2} ) - a * {t}_{2} .$$

When making the Kaplan-Meier type plot, the treatment day was recorded at the whole number day after a calculated treatment time. The average treatment day was found by averaging individual treatment days. Saline was excluded from this analysis due to the high percentage of mice that never formed tumors.

### Histology and Analysis

Tumors were explanted 15 days after inoculation, immersed in formalin for 72 hours, and then 70% ethanol for 48 hours. Tumor specimens were sliced and stained with hematoxylin and eosin, CD3, and CD31. All samples were analyzed by a blinded pathologist. For analysis of lymphocyte infiltration, 10 high-power images were taken at all areas of each tumor. Lymphocytes (stained dark brown with a clear circumference) were manually counted in each photo with the FIJI Cell-Counter plug-in.

### Power Analysis

The Statistical toolbox in Matlab was used for power calculations, specifically the sampsizepwr function with two-sided t-tests. For calculations based off experimental data, the average and distribution of the day that each formulation surpassed 100 mm² were used in the power analysis. Because 30% of the saline tumors did not form at all during the experiment, these mice were excluded from this analysis, leading the saline group to require less mice than the BME group in the power analysis. Here the effect size was calculated as an observed percent change from the formulation average of 100 mm² in 1% intervals to predict the number of mice needed. For calculations predicting the effect of the coefficient of variance on the number of mice, the variance was calculated as a percent change from 100 mm² in 1% intervals with a constant 80% power. For calculations predicting the effect of variance on the power of the study, the variance was calculated as a percent change from 100 mm² in 1% intervals with a constant 10 mouse per group sample size.

### Statistical Treatment to Find Confidence Intervals on % CV

Confidence intervals in % CV values were calculated using Vangel's modification to McKay's method, which assumes the data are approximately normal and starts giving less valuable estimates as the % CV exceed 33%. To summarize this calculation, the lower (lcl) and upper limits (ucl) were calculated as

$$lcl = \frac{K}{\sqrt{( \frac{{u}_{1} + 2}{n} - 1 ) * {K}^{2} + \frac{{u}_{1}}{n - 1}}} ,$$

$$ucl = \frac{K}{\sqrt{( \frac{{u}_{2} + 2}{n} - 1 ) * {K}^{2} + \frac{{u}_{2}}{n - 1}}} , where$$

$${u}_{1} = {χ}_{1 - α / 2 , n - 1}^{2} and$$

$${u}_{2} = {χ}_{α / 2 , n - 1}^{2} .$$

K represents a sample % CV, n is the number of samples, and χ²α represents the chi-squared distribution at the designated confidence α.

What is claimed is:

1. A tumor inoculation kit for developing a cancer model, comprising:
(a) solutions to form a self-assembled hydrogel;
(b) cancer cells; and
(c) a mixer for mixing the solutions and the cancer cells to be forming self-assembled hydrogels encapsulating the cancer cells; and
(d) a syringe for injecting the cancer cells encapsulated by the self-assembled hydrogels.

2. The kit as set forth in claim 1, wherein the solutions to form the self-assembled hydrogel are Alginate and Calcium Sulfate.

3. The kit as set forth in claim 2, wherein the Alginate ranges from 0.5 to 2.5 wt % and the Calcium Sulfate ranges from 7 mM to 15 mM or 0 mM to 15 mM.

4. The kit as set forth in claim 2, wherein the solutions to form the self-assembled hydrogel further comprise extra-cellular matrix components.

5. The kit as set forth in claim 4, wherein the extra-cellular matrix components are Laminin, Hyaluronic Acid, High Molecular Weight Hyaluronic Acid, Collagen I, Collagen II, Collagen III, Collagen IV, Collagen XVIII, Heparin, Heparin Sulfate, Fibronectin, Vitronectin, Gelatin, Elastin, Tenascin, Tenascin-C, Matrix Metalloproteinases, Basement Membrane Mixtures, or a combination thereof.

6. The kit as set forth in claim 2, wherein the solutions to form the self-assembled hydrogel further comprise growth factors.

7. The kit as set forth in claim 6, wherein the growth factors are Fibroblast Growth Factors (FGF), Vascular Endothelial Growth Factor (VEGF), Platelet-derived growth factor (PDGF), stromal-derived factor-1 (SDF-1), TGF, tumor growth factor; TNF, tumor necrosis factor, or a combination thereof.

8. The kit as set forth in claim 1, wherein the cancer cells are human or animal cancer cells.

9. The kit as set forth in claim 1, wherein the cancer cells are a mixture of different types of cancer cells.

10. The kit as set forth in claim 1, further comprising cells other than cancer cells, where the cells are Vascular Endothelial Cells, Pericytes, Adipocytes, Fibroblasts, Bone-Marrow Mesenchymal Stromal Cells, or a combination thereof.

11. The kit as set forth in claim 1, wherein the solutions form the self-assembled hydrogel with a yield stress above 10 Pa.

12. The kit as set forth in claim 1, wherein the solutions form the self-assembled hydrogel with a yield stress ranging from 20-60 Pa with a measured elastic storage modulus (G′) of 50-1000 Pa at 10 rad/s.

13. The kit as set forth in claim 1, wherein the cancer cells encapsulated by the self-assembled hydrogels together define a hydrogel volume ranging from 2 microliters to 200 microliters, wherein the cancer cells are defined by a number of cancer cells within the hydrogel volume wherein the number of cancer cells ranging from 200,000 to 1,000,000.

14. The kit as set forth in claim 1, wherein the mixer is a dual syringe mixer distinguishing two connectable syringes for fluidably mixing the solutions and the cancer cells.

15. The kit as set forth in claim 1, wherein the mixing uses a dual syringe mixer distinguishing two connectable syringes for fluidably mixing the solutions and the cancer cells.

16. A method of tumor inoculation for developing a cancer model, comprising:
(a) having solutions to form a self-assembled hydrogel;
(b) having cancer cells; and
(c) mixing the solutions and the cancer cells to be forming self-assembled hydrogels encapsulating the cancer cells; and
(d) injecting the cancer cells encapsulated by the self-assembled hydrogels into tissue.

17. The method as set forth in claim 16, wherein the solutions to form the self-assembled hydrogel are Alginate and Calcium Sulfate.

18. The method as set forth in claim 17, wherein the Alginate ranges from 0.5 to 2.5 wt % and the Calcium Sulfate ranges from 7 mM to 15 mM or 0 mM to 15 mM.

19. The method as set forth in claim 17, wherein the solutions to form the self-assembled hydrogel further comprise extra-cellular matrix components.

20. The method as set forth in claim 19, wherein the extra-cellular matrix components are Laminin, Hyaluronic Acid, High Molecular Weight Hyaluronic Acid, Collagen I, Collagen II, Collagen III, Collagen IV, Collagen XVIII, Heparin, Heparin Sulfate, Fibronectin, Vitronectin, Gelatin, Elastin, Tenascin, Tenascin-C, Matrix Metalloproteinases, Basement Membrane Mixtures, or a combination thereof.

21. The method as set forth in claim 16, wherein the solutions to form the self-assembled hydrogel further comprise growth factors.

22. The method as set forth in claim 21, wherein the growth factors are Fibroblast Growth Factors (FGF), Vascular Endothelial Growth Factor (VEGF), Platelet-derived growth factor (PDGF), stromal-derived factor-1 (SDF-1), TGF, tumor growth factor; TNF, tumor necrosis factor, or a combination thereof.

23. The method as set forth in claim 16, wherein the cancer cells are human or animal cancer cells.

24. The method as set forth in claim 16, wherein the cancer cells are a mixture of different types of cancer cells.

25. The method as set forth in claim 16, further comprising having cells other than cancer cells, where the cells are Vascular Endothelial Cells, Pericytes, Adipocytes, Fibroblasts, Bone-Marrow Mesenchymal Stromal Cells, or a combination thereof.

26. The method as set forth in claim 16, wherein the solutions form the self-assembled hydrogel with a yield stress above 10 Pa.

27. The method as set forth in claim 16, wherein the solutions form the self-assembled hydrogel with a yield stress ranging from 20-60 Pa with a measured elastic storage modulus (G′) of 50-1000 Pa at 10 rad/s.

28. The method as set forth in claim 16, wherein the cancer cells encapsulated by the self-assembled hydrogels together define a hydrogel volume ranging from 2 microliters to 200 microliters, wherein the cancer cells are defined by a number of cancer cells within the hydrogel volume wherein the number of cancer cells ranging from 200,000 to 1,000,000.

29. The method as set forth in claim 16, wherein the mixer is a dual syringe mixer distinguishing two connectable syringes for fluidably mixing the solutions and the cancer cells.

30. The method as set forth in claim 16, wherein the mixing uses a dual syringe mixer distinguishing two connectable syringes for fluidably mixing the solutions and the cancer cells.

31. The method as set forth in claim 16, further comprising adding rheological modifiers to modify rheological properties of the self-assembled hydrogel.
